(19) United States
(12) Patent Application Publication
(10) Pub. No.: US 2006/0269589 A1
(43) Pub. Date: Nov. 30, 2006

(54) Fragrance slurry pad

(76) Inventor: Sven Dobler, Fermingdale, NY (US)

Correspondence Address: Paul M. Denk;Suite 170, 763 South New Ballas Rd., St. Louis, MO, 63141, US

(21) Appl. No.: 11/438,237

(22) Filed: May 22, 2006

Related U.S. Application Data
(60) Provisional application No. 60/683,979, filed on May 24, 2005.

(51) Int. Cl. A61K 9/70

(52) U.S. Cl. 424/443

(57) ABSTRACT

The fragrance slurry pad receives a fragrance in slurry and prints the slurry into a recognizable logo, brand, or word upon a strip. To enhance the marketing impact of fragrance strip advertising and to differentiate among marketers without increase in cost, the present invention is fragrance slurry deposition pads that support and reinforce a marketing message. The preferred embodiment has a brand name, logo, or message etched into a flexographic print pad. The print pad applies the slurry to a strip located beneath a flap. Upon opening the flap, the fragrance slurry appears in a discernible pattern and visible message.

### BACKGROUND OF THE INVENTION

This fragrance slurry pad relates to sampling devices and more specifically to a pad bearing a printed fragrance sample. A unique aspect of the present pad is a fragrance sample printed in a logo, symbol, word, or mark selected by the manufacturer of the sample.

Microencapsulated fragrances have been used for decades for the purpose of sampling fragrances in both Scratch & Sniff applications and in fragrance strip applications, commonly called TruEssence® fragrance strip or Scent Strip®. The first identified strip is available from the assignee of the invention herein, Orlandi, Inc., from Farmingdale, N.Y. First, Scratch & Sniff products generally involve the application of a microencapsulated fragrance “slurry” on the surface of a coated or uncoated text or cover stock weight paper, drying the slurry by means of heated forced air, or air drying, and allowing a consumer to subsequently liberate the fragrance by scratching and breaking the capsules. Second, fragrance strip products generally involve the application of microencapsulated slurry in the folded area of a coated text weight paper, allowing the slurry to dry and adhere to the inside surfaces of the folded paper area by means of absorption and evaporation of slurry carrier material. The capsules break and liberate the fragrance upon opening the fold area of the paper. Micro-encapsulation has been well documented in the chemistry and printing literature. The carrier systems for the encapsulated fragrances are predominantly hydrous but synthetic capsules in anhydrous, solvent based systems using such materials as urea-formaldehyde, are also common in the industry.

The above mentioned fragrance sampling products can be manufactured on lithographic, flexographic or rotogravure printing machines, both in sheet or roll form, using silkscreen, flexography, extrusion and airless spray technology among other methods to apply the fragrance slurry. Manufacturers of fragrance strips have predominantly used flexographic printing technology to apply the slurry during heat-set, off-set (lithographic) web printing. Flexography involves raised printing, where the slurry is transferred from the raised surface contour of a printing plate or pad to paper stock. Plates and pads are generally made using photo-polymer technology, by etching non transfer areas away from a soft material such as rubber using mechanical, laser and other technology, die molding, casting, extruding and forming liquid, heated or resinous materials.

Initially, fragrance strip manufacturers used pads that were made to apply adhesives in the web printing industry. Over the years, very few design changes addressed functional needs of individual fragrance slurries or manufacturing problems. Prior art slurry application pad designs involve repeated patterns of diamonds, circles, hash patterns, or lines, as shown in FIG. 1.

Fragrance strips have become the predominant product used for fragrance sampling in the United States, with estimates ranging between 2.5 to 3.5 billion units annually. Fragrance strips appear in a variety of common print formats including periodical and national magazine inserts, direct mail, catalog blow-in and stitch-in inserts, billing statement enclosures, mail order envelopes and business response envelopes, and billing statement remittance envelopes. Consumers and readers generally appreciate and enjoy receiving free fragrance samples but, the competing fragrance marketing messages have become almost overwhelming. Often more than three fragrance strips are included in mail such as subscription magazines, catalogs, direct mail, and store and credit card billing statements.

The present art overcomes the limitations of the prior art. That is, in the art of the present invention, a fragrance slurry pad receives a portion of slurry upon a pad that impresses a mark, logo, or word, or marketing message upon a sampling strip.

The difficulty in providing a fragrance slurry pad is shown by the operation of a typical fragrance strip. Fragrance marketers sought for years to find new ways of sampling fragrances to differentiate their advertising message in the plethora of competing advertising. Partially in response to this need, fragrance sampling companies developed scented pressure sensitive labels marketed under such trade names such as Scent Seals®, Liquatouch®, and Discover® with the intent of incorporating them in lieu of a fragrance strip as part of the above mentioned print formats. These new label products proved ineffective and cost prohibitive and thus went abandoned in large print advertising programs.

The present invention overcomes the difficulties of the prior art. The fragrance slurry pad has a mark, word, or logo etched into a flexographic print pad. The mark, word, or logo differentiates slurry pads among the many fragrances currently upon the market. Combined with other sampling components, the fragrance slurry pad readily integrates into existing sampling programs.

### SUMMARY OF THE INVENTION

The fragrance slurry pad is a pad and method that accepts a slurry printed into a recognizable mark, logo, brand, or word, or marketing message. The present invention enhances the marketing potential and impact of fragrance strip advertising and better differentiates among marketers without any significant increase in cost. The present invention creates fragrance slurry deposition pads that support and reinforce a marketing message by having a brand name, logo, or message etched into a flexographic print pad, thus rendering the fragrance slurry in a discernible pattern upon a sampling strip and visible once the flap is opened.

### BRIEF DESCRIPTION OF THE DRAWINGS

FIGS. 1a, 1b, 1c, 1d, 1e, and 1f show a plan view of the prior art strips and various treatments to the surface of the strips;

FIGS. 2a, 2b, 2c, 2d, and 2e show a plan view of the present invention bearing various marks and logos upon the surface of strips in accordance with the present invention; and,

FIG. 3 is an isometric view of the method and apparatus for transferring the fragrance slurry to the continuous web of paper as it passes the transfer roller.

The same reference numerals refer to the same parts throughout the various figures.

### DESCRIPTION OF THE PREFERRED EMBODIMENT

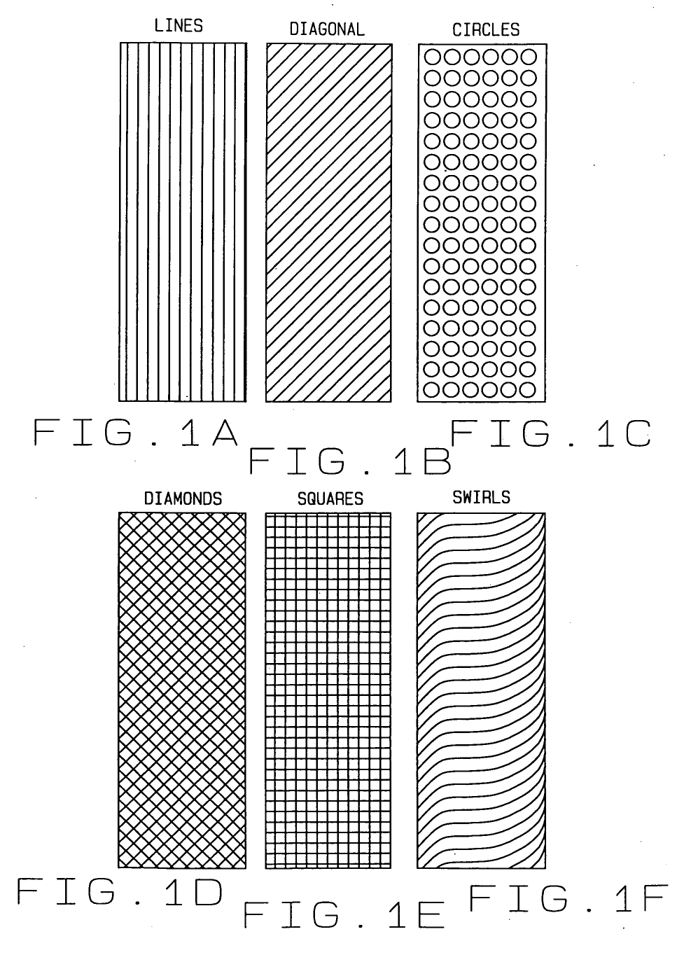

The present art overcomes the prior art limitations by use of a fragrance slurry instead of ink to be transformed from a flexographic pad to paper. Turning to FIG. 1, the prior art of fragrance strips is shown. The prior art strips bear a variety of geometric patterns applied to the surface of the strip. The geometric patterns include diagonals 1a, longitudinal lines 1b, dots 1c, diamonds 1d, and a grid 1e. The prior art patterns are applied in the same way as the current invention. A pick-up cylinder turning in a pan filled with slurry is coated by said slurry and transfers the slurry to a pad which is mounted on a transfer cylinder. The raised pad on this transfer cylinder now carries slurry and transfers the slurry by contacting a passing web of paper. The amount of slurry transferred to the paper is controlled by web tension, pressure between the web and the pad, and the speed of rotation of the pick-up cylinder on the slurry pan.

Prior art patterns on pads were driven entirely by the need to allow a variety of materials to be trapped in the contoured areas of the pad and transferred on paper. Originally, these pads were designed to apply adhesives. Beginning in the late 1970's, when flexographic technology was used to apply fragrance slurry during the web heat-set offset printing process, fragrance slurries were being applied by these pads. Slurries vary in viscosity and consistency due to the differences in fragrance oil formulations that need to be microencapsulated. In order to control and regulate the amount of slurry that could be transferred to the paper, printing companies and pad manufacturers started experimenting and modifying pad patterns.

The flexographic printing prior art is replete with different methods and technologies of creating images on print pads that transfer ink to a substrate. The point of this invention is to use colored or uncolored microencapsulated fragrance material instead of ink to print a fragranced message or image on a paper substrate.

This invention applies to other cosmetic or fragrance materials as well.

The print application of the cosmetic or fragrance material can be done with a flexographic pad in the raised area. It can also be applied in a silkscreen printing process.

The point is: using segmented or non segmented cosmetic and fragrance materials instead of ink to print a marketing message.

Many existing methods and devices place an image on paper, using ink or a die. The existing methods and devices include raised printing or flexography, offset, recessed printing or gravure, silkscreen printing or serigraphy, ink jet imaging where electrically charged pigment particles create an image in an electrostatic field, airless spraying, or air brushing often with a mask to prevent overspray. An image can also be formed by foil stamping, embossing, die-casting, and using thermoforming, vacuum forming, casting, heat treatment, electrostatic treatment, spraying, extruding, adhesives, and cohesives as methods to adhere ink or pigmented particles to a substrate in a pattern. Inks are either traditionally formulated as, offset inks, flexo inks, ultraviolet cured inks, and thermographic inks. And some printing systems require treatment ovens, or UV light to set or cure the inks or substrates. In the present invention, the inks adhere to the surface a paper substrate having a recognizable form such as a mark, word, or logo. All printing currently involves only inks, whereas, imaging a flex-o-pad in the manner as described herein, to carry a pigmented or non pigmented fragrance, in a fragrance slurry, is a new concept, and it is used in the current invention, in Scent Strip Manufacturing, to apply such through the use of a transfer pad with a discernable message.

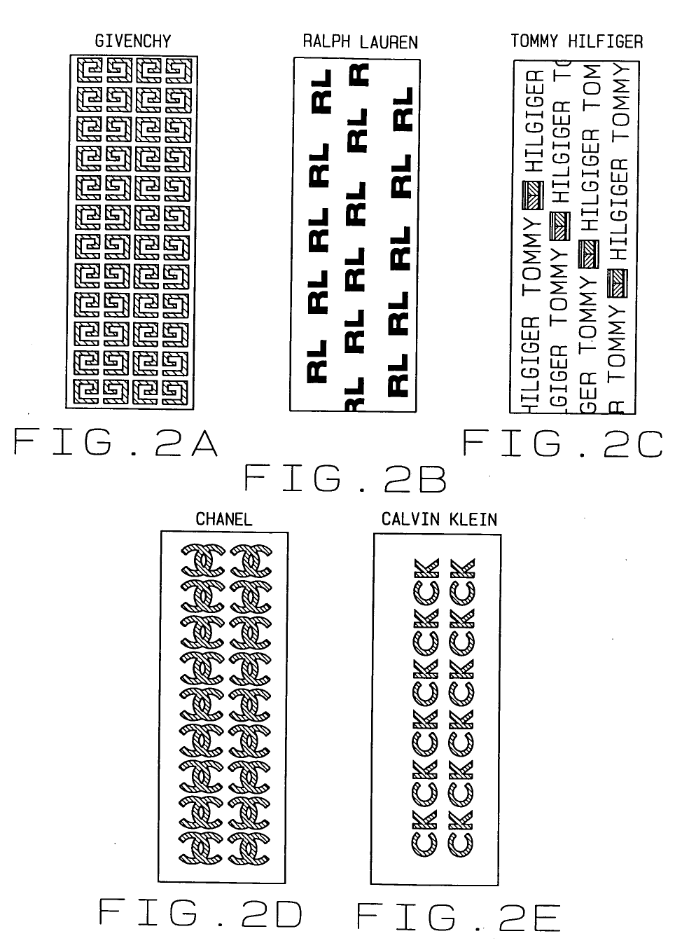

In the preferred embodiment, the present invention utilizes flexographic technology and more particularly, a laser to etch away non-image areas of a print pad. The image area of the print pad contains a pattern of a mark, word, or logo here shown repeated for the length of one or more print strips as shown in FIG. 2. Outside of the image area, the remainder area may still exhibit traditional diamond, circle, line or diagonal patterns that have effectively picked up and transferred fragrance slurry to paper in a web heat set and offset, or lithographic, printing process. In the preferred embodiment, the print pad is made of rubber. In alternate embodiment, the print pad is made of suitable ductile material.

The present invention also provides a method. Following graphic design, the present invention etches an image area into a print pad and leaves a remainder area. The image area contains the graphic design in the form of a marketing message using a mark, logo, or word. The present invention then applies a micro-encapsulated fragrance slurry to the print pad mounted in flexographic equipment. The print pad then deposits the fragrance slurry upon a paper substrate thus transferring the marketing message of the image area.

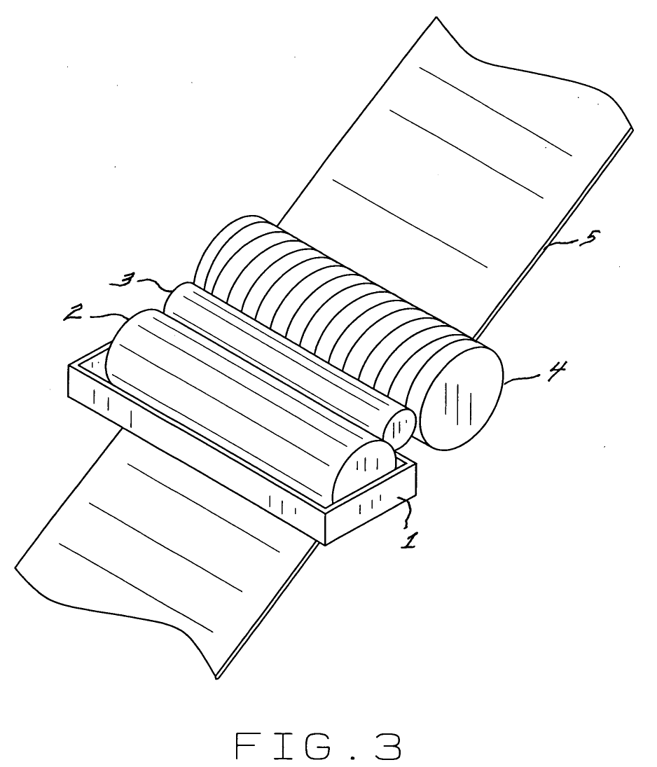

FIG. 3 shows the system and method for transferring the fragrance slurry from its pickup roller, through the transfer roller, and to the continuous web of paper. For example, the slurry tray 1 has a supply of the fragrance slurry provided therein, and a pickup roller 2 partially locates within the slurry, within the tray, to do just that, pickup a supply of the fragrance slurry for further transfer. It is then applied from the pickup roller to the transfer roller 3, and then the transfer roller passes the slurry to the flexographic print roller, at its various printing pad areas, as at 4. Then, the pattern, upon the print pad or roller, is transferred to the continuous web of paper, as at 5, as it passes the flexographic print roller. Thus, the paper is then printed with the fragrance slurry, can be dried, reconfigured into a roll form, transferred to a customer, where it may be cut into various sheets, or cards, for use for commercial purposes. The transfer roller contacts with the pads that are mounted on the flexographic print cylinder, and these pads contact the paper, to transfer the fragrance slurry thereto, into whatever discernable pattern is applied to the cylinder, such as the example of patterns as shown in FIGS. 1 and 2.

From the aforementioned description, a fragrance slurry pad has been described. The fragrance slurry pad is uniquely capable of applying a mark, word, or logo upon a fragrance strip. The fragrance slurry pad and its various components may be manufactured from many materials, including but not limited to single or in combination, rubber, polymers, polyester, polyethylene, polypropylene, polyvinyl chloride, nylon, Teslin, Saran, open cell foam, closed cell foam, ferrous and non-ferrous metal foils and their alloys, and composites.

I claim:

1. A device for applying a micro encapsulated fragrance material appearing as a discernible mark, word, or logo comprising:
a paper substrate;
a flexographic print pad, having a raised image area and a remainder area;
the fragrance material comprising a microencapsulated fragrance; wherein said microencapsulated fragrance comprises a cosmetic material including pigments and colorants, and
the cosmetic fragrance microencapsulated material being applied especially to the area of paper substrate by one of rotogravure, print cylinders, and silk screen printing, to provide an image on said paper substrate in a category of a mark, design, word, or logo for display.

2. The fragrance applying device of claim 1 wherein said printed area is raised above said remainder area and said device operates by flexography.

3. The fragrance applying device of claim 1 wherein said remainder area is recessed beneath said image for rotogravure printing.

4. The fragrance applying device of claim 1 wherein said printed area is formed by one of serigraphy or electrostatic imaging.

5. The fragrance applying device of claim 1 wherein said printed area is formed with a mask by one of airless spraying, air brushing, or extrusion.

6. The fragrance applying device of claim 1 wherein said printed area is formed without a mask by one of airless spraying, air brushing, or extrusion.

7. The fragrance applying device of claim 1 further comprising:
said fragrance slurry having colored pigments; thus, a more discernible said image area appears upon said strip.

8. The fragrance applying device of claim 1 wherein said fragrance slurry includes either powder or pigmented shell that induce a pearlescent effect.

9. The fragrance applying device of claim 1 wherein said strip is one of paper, non-woven material, or woven material.

10. A method for making a fragrance sample appear as a discernible mark, word, or logo comprising:
etching a printed area upon a print pad in a discernible mark, word, or logo, said print pad being used in flexographic printing;
leaving a remainder area upon said print pad;
applying microencapsulated fragrance slurry to said print pad;
pressing said print pad upon sampling strips; and,
packaging said sampling strips for later inspection by consumers.

11. The method for making a fragrance sample as set forth in claim 10, wherein initially supplying a tray with a supply of the fragrance slurry, partially locating a pickup roller into the trayed slurry, transferring the slurry to a transfer roller, applying the slurry to a flexographic print pad in a discernable pattern affixed onto a flexographic print cylinder, and then transferring the patterned fragrance slurry to a continuous web of paper as it passes the flexographic cylinder.
